(19) United States
(12) Patent Application Publication
(10) Pub. No.: US 2021/0299077 A1
(43) Pub. Date: Sep. 30, 2021

(54) LIPOSOMAL REDUCED GLUTATHIONE (LRG) IN COMBINATION WITH IVERMECTIN FOR THE TREATMENT OF COVID-19

(71) Applicant: Your Energy Systems, LLC, Palo Alto, CA (US)

(72) Inventor: Frederick Timothy Guilford, Palo Alto, CA (US)

(21) Appl. No.: 17/123,438

(22) Filed: Dec. 16, 2020

Related U.S. Application Data
(60) Provisional application No. 63/000,934, filed on Mar. 27, 2020.
(60) Provisional application No. 63/009,307, filed on Apr. 13, 2020.

(51) Int. Cl. A61K 31/198; A61K 9/127; A61K 31/352; A61P 31/14

(52) U.S. Cl. CPC A61K 31/198; A61P 31/14; A61K 31/352; A61K 9/127

(57) ABSTRACT

For prophylaxis and treatment of Covid-19, the invention claimed proposes a combination and associated methods of a combination of ivermectin and liposomal reduced glutathione from Your Energy Systems, LLC of Palo Alto, Calif. An alternative dosing schedule is proposed for elderly or in adult individuals with compromise of the immune system. Pedriatic doses are also specified.

For prophylaxis and treatment of Covid-19, the invention claimed proposes a combination and associated methods of a combination of ivermectin and liposomal reduced glutathione from Your Energy Systems, LLC of Palo Alto, Calif. An alternative dosing schedule is proposed for elderly or in adult individuals with compromise of the immune system.

A recently filed U.S. provisional patent application 63/000,934 filed Mar. 27, 2020 entitled Composition and Method for Restoration of Karyopherin Dysfunction Related to Covid-19 supports the use of the composition liposomal reduced glutathione for the treatment of coronavirus infection such as Covid-19 is adopted herein by reference. U.S. provisional patent application 63/009,307 filed Apr. 13, 2020 is adopted by reference.

### DESCRIPTION OF PROBLEM

Because studies show that antiparasitics in general cause an increase in oxidation stress, the problem is to enable the antiparasitics to be safer while effective and to achieve a cooperative and more effective effect by the addition of another composition that will not interfere with the antiparasitic effect and itself have qualities that interfere with viral replication and enhance normal function of a cell. Critical in the context of coronavirus is enabling and protecting macrophages that themselves can be compromised, recognizing that they are critical to combatting a virus infection Viral infections lower the pH of a cell, thereby facilitating the viral replication and also enable secondary infections, many of which are bacterial in nature. have been found to trigger oxidative stress and thus interfere with the normal function of the host cell. Therefore, the maintenance and restoration of a favorable intracellular pH environment and antioxidant supported environment is vitally important for the host to combat against virus infection. The inventor proposes the combination of liposomally formulated reduced glutathione (“liposomal reduced glutathione” or “LRG”) combined with certain antiparasitic drugs to combat coronavirus known as Covid-19.

In the quest for antioxidant protection in humans, a variety of biochemicals have been considered. For example, it has also been reported that the antioxidant effects of vitamin E are only displayed in vitro and not in vivo; thus, it remains a matter of controversy as to whether or not vitamin E is useful for protecting the body from ROS-induced oxidative stress (1).

The invention proposes to use liposomal reduced glutathione to cooperate with the antiparasitic to enable its effectiveness by diminishing viral replication, by stabilizing cell pH, deceasing damage of oxidation from the antiparasitic, while the antiparasitic can attack both the viral infected cell and cells the initially compromised cell has infected, which are compromised cells are forming syncytia that are compromising lung function. Not previously described is the buffer effect of liposomal reduced glutathione that both decreases oxidation by the antiparasitic component of the composition and method of treatment but also increases the pH of the cell because of the liposomal reduced glutathione. The purpose of this application is to present a composition consisting of liposomal reduced glutathione and an anti-parasitic medication to treat the Covid-19 virus. The combination helps slow the entry of the virus into the cell, slow the replication of virus and in combination with an antiparasitic medication helps reverses the metabolism shift that occurs with viral infection and at the same time treats cells that have undergone metabolic shift to become parasitic in their function. The total of these cooperating effects is to slow the progression of Covid-19 and allow the host system (the patient) an opportunity to recover from an infection.

Viral pathogenesis may occur in several ways (6). These include,

1. Viral infection might result in an intense inflammatory response that compromises physiological function or results in excessive destruction of host tissue. Evidence of this occurrence is the elevation of IL-6 that is reported by physicians caring for individuals with Covid-19 infection (personal unpublished communication from Dr. Manpreet Singh, St Barnabas Hospital Emergency Department, Brooklyn, N.Y. Apr. 2, 2020.)

2. A report from Germany shows Level of IL-6 predicts respiratory failure in hospitalized symptomatic COVID-19 patients https://www.medrxiv.org/content/10.1101/2020.04.01.20047381v1.full.pdf

3. Excessive production of pro-inflammatory mediators might then result in an unchecked influx of pro-inflammatory cells to the site of infection. These cells may include neutrophils and macrophages. The normal function of macrophages is required for resolution of inflammation (7) The ability of macrophages to perform their normal role in the control of infection and inflammation is compromised if glutathione is depleted.

Viruses can appropriate a range of functions in cells (8). Virus-infected cells commonly exhibit the Warburg effect: increased glycolytic metabolism in the presence of adequate oxygen for oxidative phosphorylation (9). These shifts point out the parasitic nature of viruses, which the inventor believes them responsive to treatment with antiparasitic medications although as yet no explanation has been published as why and results of administration of antimalarials, particularly hydroxychloroquine have been mixed. Increased oxidation stress on cells accompanies viral infection at all levels, from viral entry to viral replication. Studies show the antiparasitics in general cause an increase in oxidation stress. In general it appears that oxidation stress is higher during antiparasitic therapy and has been noted in the use of combinations of antiparasitic medications. The levels of MDA, a marker of oxidation were significantly higher when taking antiparasitic medications.

It has been demonstrated that acidic conditions favor many viral infections by accelerating the viral fusion process and enhancing viral replication (11) (12) (13).

Glycoproteins of COVID-19 are used by virus to attach to cells. Disulfide bonds are involved in the folding and transport of mouse hepatitis coronavirus glycoproteins (14). Proteins that do not fold properly lose their function. The amount of glutathione present during the formation and function (binding) of glycoprotein affects the formation of the glycoprotein. (15). The loss of glycoprotein protein folding in the presence of adequate glutathione explains the demographic in Covid-19 infection. The demographic of increased risk in Covid-19 includes conditions associated with low glutathione, a major marker of human disease due to its significance in maintaining redox balance throughout the body (16) and include cardiovascular diseases (17) cirrhosis of the liver (18), hypertension (19), diabetes (20) and Parkinson's disease (21). In healthy individuals with adequate availability of glutathione, either through storage or the ability to increase production, the inventor believes the infectivity of the virus is lessened. This relationship would explain why 80% of COVID-19 patients experience ‘mild’ symptoms. https://www.businessinsider.com/what-coronavirus-mild-symptoms-are-fever-2020-3 or in some instances are virtually symptom free, such as Senator and Doctor Rand Paul. The proposed solution of maintenance or restoration of glutathione and an antiparasitic drug will create cooperation between glutathione and the antiparasitic drug to slow Covid binding and support the function of the natural defenses against viral entry (22).

Once the virus is in cells oxidation in the cytoplasm and/or Endoplasmic reticulum is needed to facilitate viral replication. At this point, it becomes a race to see if a cell's biochemical machinery will be adequate to slow viral replication, or the viral replication will over take the normal cells. Microtubules support long range virion (virus particle) transport at a speed measured in μm/s (23). Inside a eukaryotic cell, most of the transmembrane and secreted proteins are translated, modified, and folded in the Endoplasmic Reticulum (ER). To avoid over loading the ER, which can cause apoptosis in cells, and which would limit viral replication, Coronavirus induces the unfolded protein response (UPR), a process that aims to restore the ER homeostasis. This effect occurs because ATF6 is pathway activated in coronavirus-infected cells, resulting in the up-regulation of chaperone proteins to counteract ER stress and continue production of viral proteins (24).

Coronaviruses have been shown to decrease the formation of glutathione by a method that is slightly different from most virus action. In addition to the utilization of cysteine by virus formation, Coronavirus produces a protein that blocks the ability of cells to increase the production of glutathione. Thus cells suffer a corollary effect of a decrease in a substance that can decrease oxidation and a consumption of a precursor, namely cysteine, to the formation of the tripeptide glutathione which consists of a glycine-cysteine and glutamine. The Covid-19 virus decreases the formation of glutathione due to block by the Covid-19 virus of karyopherin: the function of which karyopherin is to escort Nrf2 into the nucleus to trigger the formation of glutathione and related enzymes. An excess of unfolded/misfolded proteins will accumulate inside the ER and result in ER stress. In order to return to homeostasis, cells have evolved Unfolded Protein Response (UPR), which limits nascent protein transport to ER lumen (25). The IRE1 pathway, involved in the control of ER function is induced by Coronavirus and induces IL-6 (24), which induces increased oxidation stress and loss of glutathione. The consumption by the virus of cysteine, a precursor component to glutathione, favors the depletion of glutathione and attenuation of the buffer effect of glutathione, thus largely enabling stabilization in favor of the virus by creating a low pH.

Referring again to the spikes that prominently characterize coronavirus, and the protein folding in the endoplasmic reticulum, the formation of disulfide bonds are considered key structural elements for a protein's folding in the endoplasmic reticulum (“ER”) (26). The site where these bonds are formed is depleted of glutathione in a process known as “oxidative protein folding” (27). An environment rich in glutathione with its active thiol branch will inhibit the proper folding and production of these viral protein components and will unfold and disable the binding spikes of coronavirus, thereby slowing the replication of virus such as coronavirus and Covid-19.

Additionally, Coronavirus facilitates the formation of giant, multinucleated cells, or syncytia which syncytia, have been proposed as a strategy for the virus to replicate and allow direct spreading of the virus between cells, subverting virus-neutralizing antibodies.

LRG stimulates the formation of an enzyme called Paraoxonase that is upregulated in macrophage cells as PON2 (28) that can remove biofilm formation in cells as well as function n the removal of pus, an accumulation of dead cells including neutrophils that can accumulate in airways. This accumulation of syncytia and cellular debris can lead to overgrowth of bacteria and pneumonia associated with Covid-19 infection. LRG can support macrophage function that has been shown to be needed to calm inflammation from the accumulation of these materials (7).

In the case of coronavirus, efficient lytic infection produces a significant overload of viral progeny (29). Viruses change the central carbon metabolism of the infected host cells (FIG. 3), sometimes in a similar way as observed in many tumor cells, another example of parasitic transformation. These interactions activate directly or indirectly glucose uptake, glycolysis, and many other cell functions,(29). The alterations in cell function created by virus causes alteration in internal structures seen with parasites and may make viral infections susceptible to antiparasitic medications.

### DESCRIPTION OF PROPOSED SOLUTION

Current medications include
1. Hydroxychloroquine and chloroquine are oral anti-parasitic prescription drugs that used for treatment of malaria and certain inflammatory conditions.
As referenced in a French study: https://www.mediterranee-infection.com/wp-content/uploads/2020/03/Hydroxychloroquine_final_DOI_IJAA.pdf, chloroquine has been used for malaria treatment and chemoprophylaxis, and hydroxychloroquine is used for treatment of rheumatoid arthritis, systemic lupus erythematosus and porphyria cutanea tarda. Both drugs have in-vitro activity against SARS-CoV, SARS-CoV-2, and other coronaviruses, with hydroxychloroquine having relatively higher potency against SARS-CoV-2 (1). A study in China reported that chloroquine treatment of COVID-19 patients had clinical and virologic benefit versus a comparison group, and chloroquine was added as a recommended antiviral for treatment of COVID-19 in China(6). Based upon limited in-vitro and anecdotal data, chloroquine or hydroxychloroquine are currently recommended for treatment of hospitalized COVID-19 patients in several countries. A Mar. 24 2020 publication https://pubmed.ncbi.nlm.nih.gov/32211771/ relates, “Unfortunately chloroquine (CQ) did not show efficacy in inhibiting viral replication in a mouse SARS-CoV model [3]. Nevertheless, by considering its anti-inflammatory properties, it has been postulated that chloroquine and hydroxychloroquine (CQ/HCQ) may have some effect on SARS [3, 4], in particular by inhibiting the production of pro-inflammatory cytokines (TNFα, IL6) and consequently blocking the subsequent cascade of events which lead to ARDS (30). Contrary to the indication from the in vitro antiviral activity, no acute virus infection has been successfully treated by CQ/HCQ in humans [7].-CQ/HCQ by itself did not show any anti SARS-CoV effect on in-vivo model (30). Both chloroquine and hydroxychloroquine have known safety profiles with the main concerns being cardiotoxicity (prolonged QT syndrome) with prolonged use in patients with hepatic or renal dysfunction and immunosuppression.

The hydroxychloroquine clinical safety profile is better than that of chloroquine (during long-term use) and allows higher daily dose [13] and has fewer concerns about drug-drug interactions [14]. Hydroxychloroquine is efficient in clearing viral nasopharyngeal carriage of SARS-CoV-2 in COVID-19 patients in only three to six days, in most patients.

A. The composition and combined method of treatment would be first, the use of liposomal reduced glutathione available from Your Energy Systems, LLC of Palo Alto Calif. and an antiparasitic drug, particularly hydroxychloroquine sulfate, with the potential addition of azithromycin as described below.

The preferred dosing of glutathione for all of the compositions and methods is as follows:

DETERMINE INDIVIDUAL DOSE BY BODY WEIGHT: For children
Under 30 lbs: ¼-½ teaspoon=100-200 mg GSH
30-60 lbs: ½-1 teaspoon=210-420 mg GSH
60-90 lbs: ¾-1.5 teaspoon=316 mg-630 GSH
90-120 lbs: 1-2 teaspoons=422-844 mg GSH
120-150 lbs: 1½-3 teaspoons=630-1260 mg
GSH Over 150 lbs: 1½-3 teaspoons=630-1260 mg GSH

This aspect of the invention as part of the composition or the method of treatment should be used on a continuous basis during treatment.

Children—should use a dose of liposomally encapsulated reduced glutathione equivalent to 60 mg/Kg of body weight daily in divided doses.

These doses should be continued for the duration of the duration of the illness and for prevention of recurrence and to decrease the chance of shedding of virus for 2-3 weeks after the illness.

The components of this invention can be administered separately or combined in a single capsule or dose.

2 teaspoons of LRG every 2-4 hours as needed for symptoms of viral infection. After symptoms are resolved, 1 teaspoon 4 times a day should be used for 5 days and as needed to prevent recurrence of the viral infection.

As Covid-19 is more likely to occur in elderly or in adult individuals with compromise of the immune system, an increased dose from the standard glutathione support dose is recommended in the elderly population for acute conditions.

For standard support 1 teaspoon twice a day is recommended. For management of Covid-19 a dose of 2-4 teaspoons twice a day for 2-3 weeks is recommended. After that time, if the individual is suspected to have decreased glutathione and/or immune compromise a dose of 2 teaspoons twice a day may be continued.

The recommended composition and treatment would be accompanied by doses of hydroxychloroquine sulfate 200 mg, three times per day

B. An alternative composition and method of treatment with an additional drug would be

the combination of liposomal reduced glutathione, hydroxychloroquine and azithromycin

The additional dose for azithromycin would be: azithromycin (500 mg on day 1 followed by 250 mg per day, the next four days) to prevent bacterial super-infection under daily electrocardiogram control.

C. After the virus appears to have been cured, the combined composition and method of treatment would be use of liposomal reduced glutathione with dosing of liposomal reduced glutathione of 2 teaspoons every 4 hours as needed for symptoms related to viral infection. After symptoms are resolved, 1 teaspoon 4 times a day should be used for 5 days and as needed to prevent recurrence of the viral infection.

Atovaquone may be considered instead of hydroxychloroquine sulfate. Atovaquone dose is 1 tablet daily for prevention.

Atovaquone Dosage. Adult tablet: 250 mg atovaquone plus 100 mg proguanil Pediatric tablet:

62.5 mg atovaquone plus 25 mg proguanil https://www.drugs.com/dosage/malarone.html

D. An alternative composition and method of treatment with an additional drug would be the combination of liposomal reduced glutathione, atovaquone, and azithromycin The additional dose for azithromycin would be: azithromycin (500 mg on day 1 followed by 250 mg per day, the next four days) to prevent bacterial super-infection under daily electrocardiogram control.

Adults

The combined composition and method of treatment would be

2 Teaspoons of LRG every 2-4 hours until the virus is resolved. After symptoms and testing is completed to show negative for coronavirus, 1 teaspoon 4 times a day should be used for at least 5 days and as needed to prevent recurrence of the viral infection, and in terms of general immune support, can be continued without limit of time.

E. Malarone may be substituted for hydroxychloroquine sulfate or atovaquine. Four MALARONE tablets (adult strength; total daily dose 1 g atovaquone/400 mg proguanil hydrochloride) as a single daily dose for 3 consecutive days.

Pediatric Patients

The dosage for treatment of acute malaria in pediatric patients is based upon body weight (Table 2).

TABLE 2
Dosage for Treatment of Acute Malaria in Pediatric Patients

| Atovaquone/ | | |
|---|---|---|
| Proguanil | | |
| HCl | Total Daily | |
| Weight of | Dose/per dose | |
| patient (kg) | amount | Dosage Regimen |
| 5-8 | 125 mg/50 mg | 2 MALARONE pediatric |
| | | tablets daily |
| | | for 3 consecutive days |
| 9-10 | 187.5 mg/75 mg | 3 MALARONE pediatric |
| | | tablets daily |
| | | for 3 consecutive days |
| 11-20 | 250 mg/100 mg | 1 MALARONE tablet (adult |
| | | strength) daily |
| | | for 3 consecutive days |
| 21-30 | 500 mg/200 mg | 2 MALARONE tablets (adult |
| | | strength) as a single daily dose |
| | | for 3 consecutive days |
| 31-40 | 750 mg/300 mg | 3 MALARONE tablets (adult |
| | | strength) as a single daily dose |
| | | for 3 consecutive days |
| >40 | 1000 mg/400 mg | 4 MALARONE tablets (adult |
| | | strength) as a single daily dose |
| | | for 3 consecutive days |

a

2. A different approach is to use liposomal reduced glutathione with niclosamide In an individual exposed to Covid-19 or is developing symptoms of an illness with Covid-19 LRG may be administered in a dose of 2 teaspoons initially and 2 teaspoons every 2 to 4 hours until symptoms such as fever has decreased. The 2 teaspoons of liposomal reduced glutathione may be administered every 4 hours until stable in combination with Niclosamide. After symptoms are resolved, 1 teaspoon 4 times a day should be used for 5 days and as needed to prevent recurrence of the viral infection.

The dose of Niclosamide is 500 mg Three times a day. Niclosamide: Niclosamide is an FDA-approved anthelminthic drug that has been widely used in humans to treat tapeworm infections since the 1960's and is currently listed on the World Health Organization's list of essential medicines. It is inexpensive and well tolerated in vivo (32). It has been shown to have broad antiviral activities (32). The work demonstrating the efficacy of niclosamide against virus has been done with in vitro studies, and niclosamide was able to inhibit SARS-CoV replication at a micromolar concentration (33).

Niclosamide has several weaknesses such as possible cytotoxicity and limited aqueous solubility as well as relatively low absorption and oral bioavailability (F=10%), which may hamper its extensive clinical development as an antiviral agent. (32). However niclosamide was described as an old drug widely used in humans in 2004 (33).

One problem with an oral dose of niclosamide is that it is often poorly absorbed.

For instance, a single oral dose of 2000 mg of niclosamide, the maximum serum concentration of niclosamide was equivalent to 0.25-6.0 μg/mL (0.76-18.3 μM). The wide concentration range was caused by the intraindividual absorption differences. Niclosamide is only partially absorbed from the intestinal tract, and the absorbed part is rapidly eliminated by the kidneys with no cumulative toxic effects in human.(32)

The solution to increase absorption of niclosamide is to use a method of manufacturing powdered liposomal glutathione described in the a U.S. application 63/000,934 (which described the use of the composition liposomal reduced glutathione for the treatment of coronavirus infection such as Covid-19) and adapt it to the production of powdered liposomal niclosamide. In this invention, powdered liposomal niclosamide is prepared using a modified dehydration-rehydration method. One method proposed for forming powdered liposomal niclosamide is as follows: First: make a water soluble solution of niclosamide. and then making dry powders containing niclosamide-loaded liposomes (SD-niclosamide-Lip) by the following the spray-drying technique and using lactose at 10% (w/v) as drying adjuvant (Marchiori et al., 2012). Then, 10 g of lactose are dispersed in 100 mL of liposomal PBS dispersion (1 mg mL1 of the drug or blank) under magnetic stirring for 10 min. This dispersion is dried using a Mini Spray Dryer B-290 (Büchi, Switzerland) according to the following parameters: feed pump rate of 5.0 mL min⁻¹, 100% aspiration, 0.7 mm nozzle, atomization air at 819 L⁻¹ h inlet temperature 120° C. with an outlet temperature around 65 C. Maltodextrin is an alternative drying agent.

The powdered liposomal niclosamide is delivered in packets containing 7 gm of powder which delivers 500 mg niclosamide.

3. Another proposed treatment would be a composition and method of treatment utilizing Aviptadil.

For an individual ill with Covid-19, A combination of LRG and can be administered in a dose of 2 teaspoons every 1-2 hours until improvement in the patient's condition is observed in addition to the use of Aviptadil. After symptoms are resolved, 1 teaspoon 4 times a day should be used for 5 days and as needed to prevent recurrence of the viral infection.

Aviptadil, a synthetic form of Vasoactive Intestinal Polypeptide (VIP) has been awarded FDA Orphan Drug Designation for the treatment of acute respiratory distress syndrome (ARDS).

Aviptadil has been used on a compounded basis in certain ICUs for many years in the belief that it preserves life and restores function in pulmonary hypertension, ARDS, and Acute Lung Injury (ALI).

In this study, patients who are hospitalized and intubated for ARDS secondary to Covid-19 infection will be randomly allocated to Aviptadil administered by intravenous infusion, nebulization via the endotracheal tube, or both IV and endotracheal administration. Primary endpoints will be improvement in blood oxygenation and mortality.

http://www.cdc.gov/coronavirus/2019-ncov/index.html

Dose 100-200 μg dose of aviptadil by inhalation 4 times per day

https://erj.ersjournals.com/content/32/5/1289.long

intravenous infusion: 50 pmol Aviptadil/kg bodyweight/hr for 12 hrs or for a 65 kg person 130 micrograms of Aviptadil intravenous over the 12 hr infusion, or 10.8 μg Aviptadil/hr of infusion. https://patents.google.com/patent/US8178489B2/en

Patent No.: U.S. Pat. No. 8,178,489 B2

https://patentimages.storage.googleapis.com/49/08/30/a7124fb42b75d7/US8178489.pdf

4. For an individual ill with Covid-19, combination of LRG and can be administered in a dose of 2 teaspoons every 1-2 hours until improvement in the patient's condition is observed along with the use of Remdesivir. After symptoms are resolved, 1 teaspoon 4 times a day should be used for 5 days and as needed to prevent recurrence of the viral infection.

Remdesivir is an investigational intravenous drug with broad antiviral activity that inhibits viral replication through premature termination of RNA transcription and has in-vitro activity against SARS-CoV-2 and in-vitro and in-vivo activity against related betacoronaviruses. 200 mg dose of the drug on day one, followed by a 100 mg dose every day until day five or ten. https://www.clinicaltrialsarena.com/news/gilead-remdesivir-phaseii-covid-19/.

However its usefulness is not clear. Per a press release on Mar. 13, 2020: While hopes are high, RBC Capital Markets analysts examined individual patient data and decided the Gilead drug showed mixed results, at best. “Based on our review of the clinical and virological courses, we believe remdesivir's contribution to efficacy remains unclear, and with a side-effect profile that may not be completely benign,” the RBC analysts wrote in a note to investors. We continue to see a less than 50/50 possibility that the drug is ultimately proven effective.”

“More specifically, they maintained there was not a “clear temporal association” between treating patients with remdesivir and improvements in oxygen requirements, fever, and viral results, compared with hospitalized patients who did not receive the investigational drug. The analysts also noted that remdesivir patients experienced nausea, vomiting, rectal bleeding, and elevated liver enzymes.”

https://www.statnews.com/pharmlot/2020/03/13/gilead-coronavirus-covid19-clinical-trials/

The initiation of these Phase III trials comes a day after the National Institutes of Health (NIH) announced a trial of remdesivir in Covid-19 patients at the University of Nebraska Medical Center (UNMC).

5. Another well-tested anti-malarial with antibiotic properties is doxycycline which can be combined with liposomal reduced glutathione. In an individual exposed to Covid-19 or is developing symptoms of an illness with Covid-19 liposomal reduced glutathione may be administered in a dose of 2 teaspoons of liposomal reduced glutathione initially and 2 teaspoons every 2 to 4 hours until symptoms such as fever has decreased. Doxycycline can be administered with the first 2 teaspoons of liposomal reduced glutathione which thereafter may be administered every 4 hours until stable in combination with Doxycycline. After symptoms are resolved, 1 teaspoon liposomal reduced glutathione 4 times a day should be used for 5 days and as needed to prevent recurrence of the viral infection.

The Doxycycline dose is: Adults 100 mg daily. Children: >8 years old: 2.2 mg/kg (maximum is adult dose) daily and may be continued for 1-2 weeks after infection is resolved. Doxycycline may be used in place of azithromycin in situations where cardiac rhythm may be a concern.

In sum, I claim the composition, the method of treatment and the use of liposomal reduced glutathione with one of antiparasitic drugs hydroxychloroquine sulfate, chloroquine and Malrone/Atovoquone and nitazoxanide and particularly antimalarial drugs such as mefloquine, primaquine and tafenoquine.

Primaquine is also considered an antibiotic. The dose is 26.3 mg once per day (available in 30 mg tablet). Primaquine cannot be used in patients with glucose-6-phosphatase dehydrogenase (G6PD) deficiency.

Tafenoquine cannot be used in patients with glucose-6-phosphatase dehydrogenase (G6PD) deficiency. The dose is 200 mg daily for adults only. After symptoms are resolved, 1 teaspoon 4 times a day should be used for 5 days and as needed to prevent recurrence of the viral infection.

G6PD deficient limits the restoration of GSH from GSSG and is an example of the oxidation stress that antiparasitic medications may cause.

The dose for Nitazoxanide (Alina®) 300 Mg is three Times a day (TID). In addition to its antiparasitic activity, this compound has shown activity against a broad range of viruses including influenza viruses. A phase 2b/3 clinical trial (NCT01227421) aimed to determine the safety and efficacy of NTZ in the treatment of acute uncomplicated influenza established that treatment with 300 mg twice a day for 5 days was well tolerated and it was associated with a reduction of symptoms and infectious viral load (34).

We claim the addition of azithromycin to these compositions, these methods of treatment and these uses. In all combinations listed, the liposomal reduced glutathione may be in liquid or powdered form available from Your Energy Systems, LLC of Palo Alto, Calif. The liquid liposomal reduced glutathione product has approximately 422 mg per 5 ml. and the powder form when mixed with aqueous substance has 500 mg per packet.

I also claim the composition, the method of treatment and the use of liposomal reduced glutathione with drugs more generally referred to as antivirals: Aviptadil, Remdesivir, and Ivermectin. The dose of Ivermectin for adults is 12 mg three times a day. That dose can be reduced for pediatric patients according to proper pharmaceutical practice.

I also claim the composition, the method of treatment and the use of liposomal reduced glutathione with niclosamide as described, along with the potential addition of azithromycin in the doses recommended above.

Aviptadil, a synthetic form of Vasoactive Intestinal Polypeptide (VIP) can be added to the method of treatment to improve lung function.

If not otherwise described in this application, the dosing for these drugs is according to the package insert approved by the Food and Drug Administration of the United States.

### EXAMPLES

### Example 1

An elderly adult lives in a house with a young child who is experiencing a viral upper respiratory infection, who presents to the emergency department with symptoms of a cough for 24 hours and received a diagnosis that COVID19 is suspected as there is no sign of bacterial infection would be tested. Testing is obtained but would take 2-5 days to become available. The physician would elect to initiate treatment with the composition described, liposomal reduced glutathione+one of the listed medications. In this example niclosamide will be the second component. The dosing for adults with a chronic condition is 1 teaspoon (420 mg glutathione) liquid or 1 packet powder twice a day the LSRG referred to in this application if available from Your Energy Systems, of Palo Alto, Calif. in liquid or powder form. In an acute situation such as this, additional doses may be considered and the physician recommends 2 teaspoons every 6 hours for 24 hours to support glutathione, which may be reduced in this condition. It is known that plasma glutathione levels are decreased in aging (4). The second component, niclosamide is dosed at 500 mg 3 times a day. The testing for Covid-19 subsequently would be predicted to turn positive.

Children would be treated with ½ the adult dose at 1 teaspoon of liquid or 1 packet of powder liposomal glutathione every 6 hours for 24 hours to support glutathione. The second component, niclosamide is dosed at 250 mg 3 times a day.

Example 2 An adult in his 40s experiencing a viral upper respiratory infection, would be presented to the emergency department with symptoms of a cough for over 24 hours. The individual's respiratory ability is likely beginning to be compromised although his Fingertip Pulse Oximeter Blood Oxygen Monitor level of pO₂ testing is 96%, decreased slightly but normal, Covid-19 testing is obtained but will take 2-5 days to become available.

The physician would initiate treatment with the composition described, liposomal reduced glutathione+one of the treatments 1-5. In this example, hydroxychloroquine sulfate 200 mg, three times per day is elected. The physician would recommend 2 teaspoons (10 cc or 10 ml) of the liquid liposomal reduced glutathione initially and 1-2 teaspoons every 4-6 hours for 24 hours to support glutathione, which may be reduced in this condition. Additional doses may be considered.

The physician may also chose to use the powdered liposomal glutathione disclosed in this application and would use 1 or 2 packets of powder providing 500 mg each packet of glutathione initially and 1 or 2 packets every 4-6 hours. Additional dosing may be considered, as needed.

The invention is not meant to be limited to the disclosures, including best mode of invention herein, and contemplates all equivalents to the invention and similar embodiments to the invention for humans and mammals and veterinary science. Equivalents include all pharmacologically active racemic mixtures, diastereomers and enantiomers of the listed compounds and their pharmacologically acceptable salts in any pharmaceutically acceptable carrier.
1. Zhang Z, Rong L, Li Y P. Flaviviridae Viruses and Oxidative Stress: Implications for Viral Pathogenesis. Oxid Med Cell Longev. 2019; 2019:1409582. PMCID: PMC6720866 publication of this paper. https://www.ncbi.nlm.nih.gov/pubmed/31531178
6. Perlman S, Dandekar A A. Immunopathogenesis of coronavirus infections: implications for SARS. Nat Rev Immunol. 2005; 5(12):917-27. https://pubmed.ncbi.nlm.nih.gov/16322745 https://www.ncbi.nlm.nih.gov/pmc/articles/PMC7097326/7.
7. Gamage L N, Charavaryamath C, Swift T L, Singh B. Lung inflammation following a single exposure to swine barn air. J Occup Med Toxicol. 2007; 2:18. PMCID: 2234408. http://www.ncbi.nlm.nih.gov/pmc/articles/PMC2234408/pdf/1745-6673-2-18.pdf
8. Tessier T M, Dodge M J, Prusinkiewicz M A, Mymryk J S. Viral Appropriation: Laying Claim to Host Nuclear Transport Machinery. Cells. 2019; 8(6). PMCID: PMC6627039. https://www.ncbi.nlm.nih.gov/pubmed/31181773
9. Thaker S K, Ch′ng J, Christofk H R. Viral hijacking of cellular metabolism. BMC Biol. 2019; 17(1):59. PMCID: PMC6637495. https://www.ncbi.nlm.nih.gov/pubmed/31319842
11. Sarkar D P, Morris S J, Eidelman O, Zimmerberg J, Blumenthal R. Initial stages of influenza hemagglutinin-induced cell fusion monitored simultaneously by two fluorescent events: cytoplasmic continuity and lipid mixing. J Cell Biol. 1989; 109(1):113-22. http://www.ncbi.nlm.nih.gov/entrez/query.fcgi?cmd=Retrieve&db=PubMed&dopt=Citation&list_uids=2745545
12. Perez L, Carrasco L. Involvement of the vacuolar H(+)-ATPase in animal virus entry. J Gen Virol. 1994; 75 (Pt 10):2595-606. http://www.ncbi.nlm.nih.gov/entrez/query.fcgi?cmd=Retrieve&db=PubMed&dopt=Citation&list_uids=7931146
13. Ozawa M, Asano A, Okada Y. Importance of interpeptide disulfide bond in a viral glycoprotein with hemagglutination and neuraminidase activities. FEBS Lett. 1976; 70(1):145-9. http://www.ncbi.nlm.nih.gov/entrez/query.fcgi?cmd=Retrieve&db=PubMed&dopt=Citation&list_uids=186316
14. Opstelten D J, de Groote P, Horzinek M C, Vennema H, Rottier P J. Disulfide bonds in folding and transport of mouse hepatitis coronavirus glycoproteins. J Virol. 1993; 67(12):7394-401. PMCID: PMC238203. https://www.ncbi.nlm.nih.gov/pubmed/8230460
15. Bosello-Travain V, Conrad M, Cozza G, Negro A, Quartesan S, Rossetto M, et al. Protein disulfide isomerase and glutathione are alternative substrates in the one Cys catalytic cycle of glutathione peroxidase 7. Biochim Biophys Acta. 2013; 1830(6): 3846-57. https://www.ncbi.nlm.nih.gov/pubmed/23454490
16. Teskey G, Abrahem R, Cao R, Gyurjian K, Islamoglu H, Lucero M, et al. Glutathione as a Marker for Human Disease. Adv Clin Chem. 2018; 87:141-59. https://www.ncbi.nlm.nih.gov/pubmed/30342710
17. Patel R S, Ghasemzadeh N, Eapen D J, Sher S, Arshad S, Ko Y A, et al. Novel Biomarker of Oxidative Stress Is Associated With Risk of Death in Patients With Coronary Artery Disease. Circulation. 2016; 133 (4): 361-9. PMCID: 4722941. http://www.ncbi.nlm.nih.gov/pubmed/26673559
18. Loguercio C, Taranto D, Vitale L M, Beneduce F, Del Vecchio Blanco C. Effect of liver cirrhosis and age on the glutathione concentration in the plasma, erythrocytes, and gastric mucosa of man. Free Radic Biol Med. 1996; 20(3):483-8. http://www.ncbi.nlm.nih.gov/entrez/query.fcgi?cmd=Retrieve&db=PubMed&dopt=Citation&list_uids=8720922
19. Fink K, Duval A, Martel A, Soucy-Faulkner A, Grandvaux N. Dual role of NOX2 in respiratory syncytial virus- and sendai virus-induced activation of NF-kappaB in airway epithelial cells. J Immunol. 2008; 180(10):6911-22. https://www.ncbi.nlm.nih.gov/pubmed/18453612
20. Lagman M, Ly J, Saing T, Kaur Singh M, Vera Tudela E, Morris D, et al. Investigating the Causes for Decreased Levels of Glutathione in Individuals with Type II Diabetes. PLoS One. 2015; 10(3):e0118436. http://www.ncbi.nlm.nih.gov/pubmed/25790445
21. Zeevalk G D, Razmpour R, Bernard L P. Glutathione and Parkinson's disease: is this the elephant in the room? Biomed Pharmacother. 2008; 62(4):236-49. http://www.ncbi.nlm.nih.gov/entrez/query.fcgi?cmd=Retrieve&db=PubMed&dopt=Citation&list_uids=18400456
22. Jenssen H, Hamill P, Hancock R E. Peptide antimicrobial agents. Clin Microbiol Rev. 2006; 19(3):491-511. PMCID: PMC1539102. https://www.ncbi.nlm.nih.gov/pubmed/16847082
23. Wang I H, Burckhardt C J, Yakimovich A, Greber U F. Imaging, Tracking and Computational Analyses of Virus Entry and Egress with the Cytoskeleton. Viruses. 2018; 10(4). PMCID: PMC5923460. https://www.ncbi.nlm.nih.gov/pubmed/29614729
24. Fung T S, Liu D X. Coronavirus infection, ER stress, apoptosis and innate immunity. Front Microbiol. 2014; 5:296-. https://pubmed.ncbi.nlm.nih.gov/24987391 https://www.ncbi.nlm.nih.gov/pmc/articles/PMC4060729/25.
25. Fung T S, Liao Y, Liu D X. Regulation of Stress Responses and Translational Control by Coronavirus. Viruses. 2016; 8(7). PMCID: PMC4974519. https://www.ncbi.nlm.nih.gov/pubmed/27384577
26. van Anken E, Sanders R W, Liscaljet I M, Land A, Bontjer I, Tillemans S, et al. Only five of 10 strictly conserved disulfide bonds are essential for folding and eight for function of the HIV-1 envelope glycoprotein. Mol Biol Cell. 2008; 19(10):4298-309. PMCID: PMC2555952. https://www.ncbi.nlm.nih.gov/pubmed/18653472
27. Smirnova O A, Bartosch B, Zakirova N F, Kochetkov S N, Ivanov A V. Polyamine Metabolism and Oxidative Protein Folding in the ER as ROS-Producing Systems Neglected in Virology. Int J Mol Sci. 2018; 19(4). PMCID: PMC5979612. https://www.ncbi.nlm.nih.gov/pubmed/29673197
28. Rosenblat M, Volkova N, Coleman R, Aviram M. Anti-oxidant and anti-atherogenic properties of liposomal glutathione: studies in vitro, and in the atherosclerotic apolipoprotein E-deficient mice. Atherosclerosis. 2007; 195(2):e61-8. http://www.ncbi.nlm.nih.gov/pubmed/17588583
29. Eisenreich W, Rudel T, Heesemann J, Goebel W. How Viral and Intracellular Bacterial Pathogens Reprogram the Metabolism of Host Cells to Allow Their Intracellular Replication. Front Cell Infect Microbiol. 2019; 9:42. PMCID: PMC6409310. https://www.ncbi.nlm.nih.gov/pubmed/30886834
30. Guastalegname M, Vallone A. Could chloroquine/hydroxychloroquine be harmful in Coronavirus Disease 2019 (COVID-19) treatment? Clin Infect Dis. 2020.
31. Xu T L, Han Y, Liu W, Pang X Y, Zheng B, Zhang Y, et al. Antivirus effectiveness of ivermectin on dengue virus type 2 in Aedes albopictus. PLoS Negl Trop Dis. 2018; 12(11):e0006934. PMCID: PMC6277121. https://www.ncbi.nlm.nih.gov/pubmed/30452439
32. Xu J, Shi P Y, Li H, Zhou J. Broad Spectrum Antiviral Agent Niclosamide and Its Therapeutic Potential. ACS Infect Dis. 2020. PMCID: PMC7098069. https://www.ncbi.nlm.nih.gov/pubmed/32125140
33. Wu C J, Jan J T, Chen C M, Hsieh H P, Hwang D R, Liu H W, et al. Inhibition of severe acute respiratory syndrome coronavirus replication by niclosamide. Antimicrob Agents Chemother. 2004; 48(7):2693-6. PMCID: PMC434198. https://www.ncbi.nlm.nih.gov/pubmed/15215127
34. Behzadi M A, Leyva-Grado V H. Overview of Current Therapeutics and Novel Candidates Against Influenza, Respiratory Syncytial Virus, and Middle East Respiratory Syndrome Coronavirus Infections. Front Microbiol. 2019; 10:1327. PMCID: PMC6594388. https://www.ncbi.nlm.nih.gov/pubmed/31275265

I claim:

1. A composition administered as a prophylactic against COVID-19, comprising:
liposomal reduced glutathione and ivermectin.

2. A composition to treat COVID-19 comprising:
liposomal reduced glutathione and ivermectin.

3. The composition according to claim 2, further comprising:
12 mg of ivermectin three times a day, and at least ten milliliters of liposomal reduced glutathione per day or at least two packets of liposomal reduced glutathione per day.

4-8. (canceled)
